(19) United States
(12) Patent Application Publication
(10) Pub. No.: US 2024/0023631 A1
(43) Pub. Date: Jan. 25, 2024

(54) ELECTRONIC VAPORIZER AND CONTROL METHOD

(71) Applicant: EVOLV, LLC, Ashtabula, OH (US)

(72) Inventor: John Bellinger DECKER, Pittsburgh, PA (US)

(21) Appl. No.: 17/621,671

(22) PCT Filed: Jun. 22, 2020
(86) PCT No.: PCT/US2020/039024

§ 371 Date: Jul. 2, 2023

Related U.S. Application Data
(60) Provisional application No. 62/864,659, filed on Jun. 21, 2019.

(51) Int. Cl. A24F 40/57; A24F 40/10; A24F 40/51; H05B 1/02

(52) U.S. Cl. CPC A24F 40/57; A24F 40/10; A24F 40/51; H05B 1/0244

(57) ABSTRACT

An electronic vaping device comprises a heating element that is to be energized to convert a portion of a medium into a vapor by elevating a temperature of the medium, which comprises at least a first chemical constituent to be included in the vapor. Air entraining the vapor flows through an airflow passage as a result of a user inhaling through a mouthpiece during a puff. A sensor is arranged to sense a parameter indicative of an airflow rate of the air entraining the vapor flowing through the airflow passage. A controller controls operation of the heating element based on the airflow rate of the air indicated by the sensed parameter, controlling at least one of a concentration and/or a yield of the first chemical constituent, and/or a temperature of the vapor flowing through a mouthpiece based on the airflow rate indicated by the parameter sensed by the sensor.

### BACKGROUND OF THE INVENTION

### 1. Field of the Invention

This application relates generally to an electronic vaporizer and control method that varies a concentration of a component in a vaping liquid in an inhaled airstream that simulates a component profile of a traditional tobacco cigarette.

### 2. Description of Related Art

Traditional tobacco cigarettes offer a yield that increases as a function of airflow being drawn through the cigarette. The yield can indicate a quantity of one or more chemical constituents such as nicotine, for example, or a total quantity of smoke output by drawing air through the cigarette. Generally, as more air is drawn through the cigarette, the higher the yield realized by the smoker, up to a physical limit of the cigarette and a practical limit to the amount of air drawn through the cigarette.

The concentration of one or more chemical constituents such as nicotine, for example, in the smoke produced by a cigarette, or the total quantity of the smoke is initially low at the beginning of a puff Increased combustion of the tobacco is initiated at the start of the puff, as more oxygen begins to be drawn through the cigarette relative to a time when the cigarette is at rest (i.e., when a puff is not being performed). Combustion is rapidly accelerated, and the concentration increases as the airflow increases during the initial stages of the puff. As combustion of the tobacco approaches a combustion limit during the puff, the concentration of the chemical constituent produced by the cigarette levels off, even if the airflow continues to increase. Drawing more air through the cigarette than required to reach the combustion limit results in the rate at which the cigarette can produce the chemical constituent(s) being surpassed by the rate at which air is drawn through the cigarette. As a result, the chemical constituent becomes diluted in the air being drawn through the cigarette.

Due to the characteristics of tobacco cigarettes, people who have smoked for prolonged periods of time are accustomed to experiencing lower yields at the beginning of a puff, when airflow is relatively low. As airflow increases, the yield also increases as a result of increased tobacco combustion and the increased inhalation of the chemical constituent(s). The concentration of the chemical constituent(s) increases as the airflow initially increases, rapidly approaching an upper concentration limit before leveling off. The result is that smokers desiring to lessen the yield and/or concentration of the chemical constituent(s) inhaled have become accustomed to decreasing the airflow drawn through the cigarette or, in other words, taking a weak puff.

In contrast, electronic vaping devices operate to supply a generally constant yield over a range of different airflows commonly established during use of the electronic vaping device. Thus, the yield of the chemical constituent at relatively-low airflows is the same as the yield for relatively-high airflows. A constant yield is unfamiliar to long-time smokers.

For example, a user may want to take a weak puff (i.e., establish a relatively-low airflow) using an electronic vaping device in an attempt to satisfy a desire for a mild-tasting or low-yield of the chemical constituent(s). A long-time smoker familiar with the dynamics of cigarettes, however, will intuitively inhale slowly during a puff using an electronic vaping device. Because of the constant yield produced by electronic vaping devices, the electronic vaping device supplies the user with the same yield that is produced for relatively-high airflows. Since the chemical constituent(s) is/are not significantly diluted in a large quantity of air being inhaled, the end result is a highly-concentrated, or strong-tasting puff, which is the opposite of the puff desired by the user. The unexpected amount and/or concentration of the chemical constituent(s) creates an unpleasant and unfamiliar experience for the user, interfering with widespread adoption of the electronic vaping device by long-time smokers.

### BRIEF SUMMARY OF THE INVENTION

Accordingly, there is a need in the art for an electronic vaping device and control method that generates an aerosol (e.g., a vapor) according to a chemical constituent(s) profile that is familiar to smokers of tobacco cigarettes.

According to one aspect, the subject application involves an electronic vaping device comprising a heating element that is to be energized to convert a portion of a liquid into a vapor by elevating a temperature of the liquid. The liquid comprises at least a first chemical constituent to be included in the vapor. The electronic vaping device includes an airflow passage through which air entraining the vapor flows as a result of a user inhaling through a mouthpiece during a puff. A sensor is arranged to sense a parameter indicative of an airflow rate of the air entraining the vapor flowing through the airflow passage. A controller controls operation of the heating element based on the airflow rate of the air indicated by the sensed parameter, controlling a concentration of the first chemical constituent based on the airflow rate indicated by the parameter sensed by the sensor.

According to another aspect, the subject application involves an electronic vaping device that comprises a heating element to be energized to convert a portion of a liquid into a vapor by elevating a temperature of the liquid. The liquid comprises at least a first chemical constituent to be included in the vapor. The electronic vaping device also includes an airflow passage through which air entraining the vapor flows as a result of a user inhaling through a mouthpiece during a puff. A sensor is arranged to sense a parameter indicative of an airflow rate of the air entraining the vapor flowing through the airflow passage. Control circuitry controls operation of the heating element based on the airflow rate of the air indicated by the sensed parameter, increasing a yield of the first chemical constituent in the vapor entrained in the air as a result of an increase in the airflow rate over a relatively-low range of flow rates of the air flowing through the airflow passage.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 is an embodiment of a curve graphically depicting a relationship between a yield of one or more inhaled components of cigarette smoke, and the airflow rate during a puff performed on a tobacco cigarette;

FIG. 2 is an embodiment of a curve graphically depicting a relationship between a concentration of one or more inhaled components of cigarette smoke, and the airflow rate during a puff performed on a tobacco cigarette;

FIG. 3 is an embodiment of a curve graphically depicting a constant relationship between a yield of one or more inhaled components of a vapor, and the airflow rate during a puff performed on a vaping device;

FIG. 4 is an embodiment of a curve graphically depicting a relationship between a concentration of one or more inhaled components of a vapor, and the airflow rate during a puff performed on a vaping device;

FIG. 5 is an embodiment of a curve graphically depicting a specific relationship between a yield of one or more inhaled components of a vapor, and the airflow rate during a puff performed on an electronic vaping device with an output power of approximately 12 W;

FIG. 6 is a partially-cutaway view of an illustrative embodiment of an electronic vaping device that includes a sensor operatively connected to a control system for producing a yield and/or concentration of a chemical constituent of a vapor that simulates a yield and/or concentration of a combustible tobacco cigarette, respectively;

FIG. 7 is a flow diagram graphically depicting a process of controlling operation of a heating element of an electronic vaping device to generate a desired concentration v. airflow rate profile;

FIG. 8 shows a concentration profile of vapor concentration in mg per mL of air generated according to the present disclosure;

FIG. 9 shows a graphical comparison of a vapor profile produced by a combustible tobacco cigarette, a conventional vaping device, and the present electronic vaping device, with a controller as described herein;

FIG. 10 shows a yield surface plot for an illustrative example of the present electronic vaping device, at draw speeds producing airflows from 10 to 30 mL/second, and at operational powers from 10 to 15 watts; and

FIG. 11 shows a simplified relationship between an output power supplied to a heating element of an electronic vaping device for a range of different airflow rates.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

Significant time and effort have been expended in making electronic vaping device, interchangeably referred to herein as e-cigarettes, produce a consistent yield of one or more chemical constituents across a variety of parameters. Originally, the resistive heating element was directly connected to the battery, resulting in a constant power output. Voltage-controlled e-cigarettes caused the output yield per puff to remain consistent despite changes in battery charge state. Wattage controlled e-cigarettes made the output yield consistent despite changes in atomizer resistance.

However, significant numbers of current and former smokers have tried e-cigarettes, only to fail to adopt them. A persistent complaint is that it the sensation of electronic vaping devices “isn't like a traditional tobacco cigarette,” or the unfamiliar output of the electronic vaping device “makes me cough.” Users that transition successfully have had to re-learn how to draw on the devices, with the most common advice being to “draw smoothly,” or take a consistent puff (e.g., draw air through the electronic vaping device at a constant flow rate).

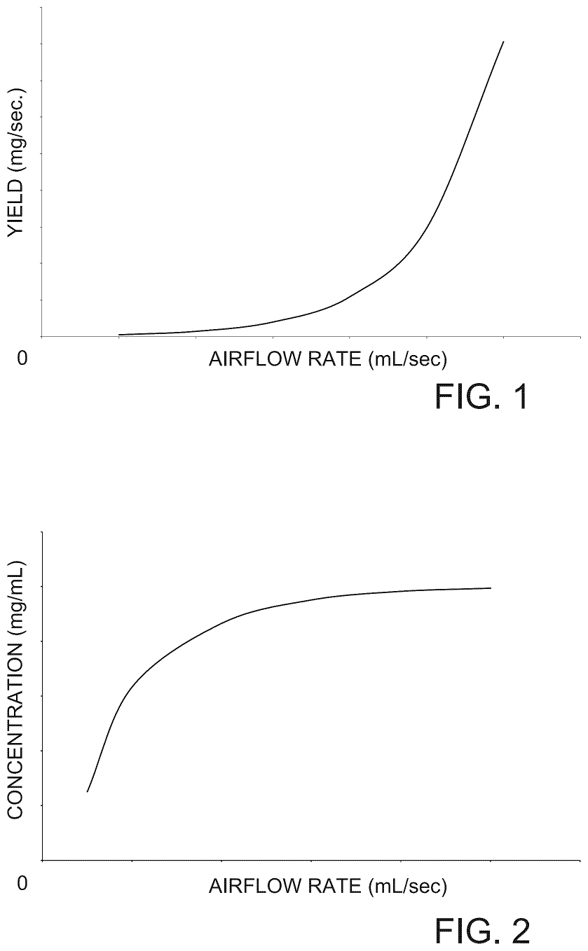

FIG. 1 is an embodiment of a curve graphically depicting a general relationship between a yield of one or more inhaled components of cigarette smoke, or the quantity of smoke, and the airflow rate during a puff performed on a tobacco cigarette. Combustible, tobacco cigarettes have a burning coal at the lit tip, through which air is drawn during a puff. Burning of the tobacco directly creates smoke, while also heating the incoming air. As the puff progresses, the smoke and heated incoming air passes over the remainder of the tobacco between the burning coal and the filter (or end for filter-less cigarettes), partially pyrolyzing the tobacco to create additional smoke. The combustible cigarette shows an increase in output yield with increasing rates of airflow. Embodiments of the electronic vaping device and control methods described herein can cause the electronic vaping device to produce a yield curve resembling the yield curve of a tobacco cigarette.

The yield and concentration are described herein with reference to a “chemical constituent.” With regard to smoke from a tobacco cigarette, the chemical constituent can be a specific chemical component, among a plurality of different chemical constituents in the smoke, or the total quantity of smoke. Similarly, with regard to a vapor (interchangeably referred to herein as an aerosol) produced by an electronic vaping device, the “chemical constituent” can refer to a specific chemical component, among a plurality of different chemical constituents in the vapor, or the total quantity of the vapor produced.

When the airflow rate (arranged along the abscissa in FIG. 1) is relatively low, the yield (arranged along the ordinate) of the chemical constituent is relatively low. As the airflow rate increases, the yield also increases at a rate that is believed to be somewhat exponential in nature. Thus, at relatively-low airflow rates, the slope of the yield curve is believed to be a low positive value, flat, or even a low negative value, before increasing with the increasing airflow rate. This slope is referred to herein as being “substantially flat” at relatively-low airflow rates (e.g., up to 30 mL/sec., or up to 40 mL/sec., etc.) for convenience. Although the curve in FIG. 1 has a somewhat exponential shape, the yield of a combustible tobacco cigarette can be substantially linear. For example, between airflow rates from about 10 mL/sec to about 40 mL/sec, the yield can linearly increase from about 0.4 mg/sec. to about 4.2 mg/sec. Generally, the yield of a combustible cigarette increases with increasing airflow rates over an operational range of airflow ranges. Notably, though, the combustible cigarette's yield per second is believed to be controlled as a function of the rate at which air is drawn and flows through the cigarette.

FIG. 2 is an embodiment of a curve graphically depicting a general relationship between a concentration of one or more chemical constituents being inhaled as cigarette smoke (along the ordinate), and the airflow rate (along the abscissa) during a puff performed on a tobacco cigarette. Embodiments of the electronic vaping device and control methods described herein can cause the electronic vaping device to produce a similar concentration curve.

When the airflow rate (arranged along the abscissa in FIG. 2) is relatively low, the concentration of the chemical constituent is relatively low. As the airflow rate increases, the concentration rapidly increases, and then begins to level off at relatively-high airflow rates, resembling an inverse exponential pattern. Thus, at relatively-low airflow rates, the slope of the yield curve is a high positive value, that rapidly decreases with the increasing airflow rate.

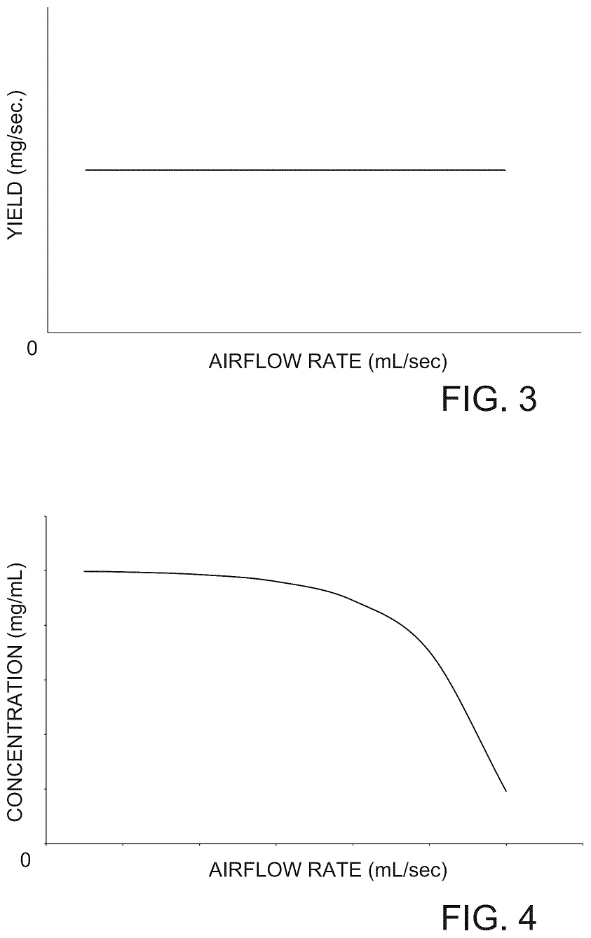

In contrast, FIGS. 3 and 4 are general representations of the relationship of the yield and concentration, respectively, of a chemical constituent to airflow rates for conventional electronic vaping devices. As shown, the yield is substantially flat over the range of airflow rates, which can include relatively-low airflow rates over the full range of standard operational airflow rates commonly encountered by electronic vaping devices. Thus, the slope of the yield curve in FIG. 3 is substantially constant, with a low value approaching zero, or up to ±2.

The generic curve in FIG. 4 is indicative of a relationship between the concentration of the chemical constituent to airflow rates having a substantially flat slope over a range of relatively-low airflow rates, that rapidly decreases, approaching a high negative value.

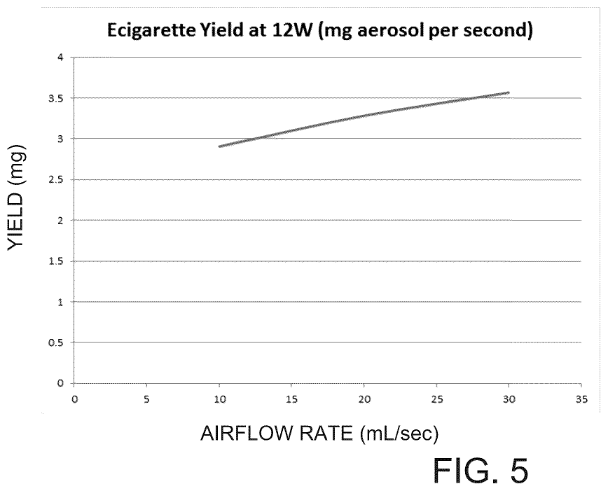

As a specific example, FIG. 5 shows the yield (e.g., milligrams of aerosol per second) of a standard e-cigarette at 12 W, at flow rates of the aerosol from 10 mL/sec to 30 mL/sec, which is a typical usage range for this particular device. An example of a limiting factor governing the airflow rates is the draw resistance, which is indicative of a difficulty a user experiences in inhaling the vapor through the electronic vaping device. A lack of draw resistance due to the inlet orifice size makes it difficult for a user to inhale more slowly than 10 mL/sec, and excessive draw resistance makes it difficult to inhale faster than 30 mL/sec. These values are device-specific, and are provided for illustrative purposes only.

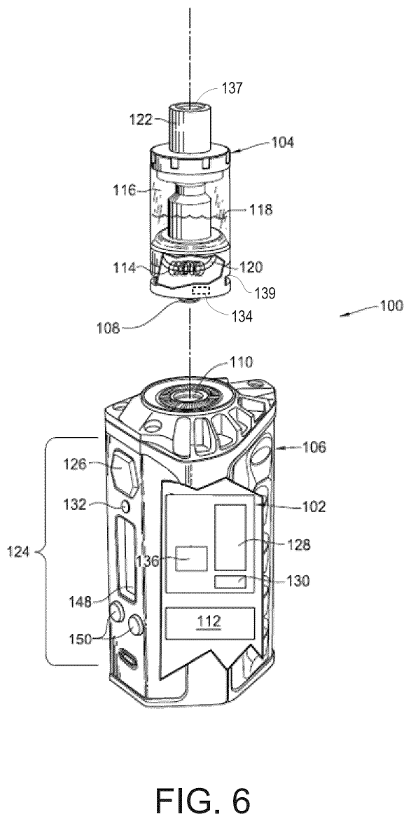

FIG. 6 shows a partially-cutaway view of an illustrative embodiment of an electronic vaping device 100 that produces a yield and/or concentration profile for a chemical constituent of a vapor, simulating a yield and/or concentration, respectively, of a combustible tobacco cigarette. The illustrated embodiment includes a control circuit 102, interchangeably referred to herein as a controller 102, for reproducing a puff based on a yield and/or concentration profile stored in a non-transitory, computer-readable medium 130 (“CRM 130”). According to some embodiments, the controller 102 can further control operation of the heating element 114 based on the airflow rate of the air indicated by the sensed parameter to at least maintain a temperature of the vapor flowing through the mouthpiece 122 as a flow rate of the air flowing through the airflow passage increases over the relatively-low range of airflow rates.

The electronic vaping device includes a tank 104, also referred to as an atomizer, that is releasably coupled to a vaporizer body 106. The tank 104 is removable, and capable of being re-installed on the vaporizer body 106 or replaced by a compatible replacement tank. The tank 104 includes a first connector portion 108 (e.g., a male threaded member in FIG. 1) that cooperates with a second connector portion 110 (e.g., a female threaded receiver in FIG. 1) to install the tank 104 on the vaporizer body 106 in a removable manner, but other releasable/re-installable connectors can be utilized. For example, compatible twist-lock fastener components, or any other releasable connector components can be utilized to allow for the installation of the tank 104 onto the vaporizer body 106 and the removal of the tank 104 from the vaporizer body 106.

The first and second connector portions 108, 110 can collectively form an electrical connector that establishes an electrical connection between the tank 104 and the vaporizer body 106. Output power can be supplied from a battery 112 or other power source provided to the vaporizer body 106 to electric components such as a heating element 114 provided to the tank 104 as described in detail herein. An example of the battery 112 includes, but is not limited to a rechargeable, lithium-ion battery, for example, but other energy sources are also contemplated by the present disclosure.

The tank includes a reservoir 116 that stores the e-liquid 118 or other medium that is to be at least partially converted into a vapor as described herein. Although embodiments of the medium are described herein as a liquid that is at least partially converted into a vapor for illustrative purposes, other embodiments of the medium can include a wax based material, leafy organic material, gel, and any other media that, when heated by the heating element 114, is at least partially converted into a vapor. According to some embodiments utilizing the e-liquid 118, wicking material 120 is arranged in fluid communication with the e-liquid 118 in the reservoir 116, and positioned adjacent to the heating element 114. The wicking material 120 conveys the e-liquid 118 from the reservoir 116 to the heating element. Activation of the heating element 114 as described herein elevates a temperature of a portion of the e-liquid conveyed by the wicking material 120, converting the portion of the e-liquid 118 into a vapor.

The term “vapor,” as used herein, refers to gaseous molecules of the e-liquid 118 that are evaporated, and small liquid droplets of the e-liquid 118 that are to be suspended or entrained in the air flowing through the electronic vaping device 100 as an aerosol, as a result of being exposed to an elevated temperature of a heating element 114 provided to the tank 104. It is the vapor entrained in the air that is inhaled by a user of the electronic vaporizer through a mouthpiece 122 provided to the tank 104 of the illustrative embodiment appearing in FIG. 6.

The embodiment of FIG. 6 shows the tank 104 as being removable from the vaporizer body 106. However, it is to be understood that other embodiments of the electronic vaping device can include a permanently-installed tank that is formed as an integral component that is fixed to the vaporizer body, and is not removable from the vaporizer body without damaging the electronic vaporizer. Such an electronic vaporizer configuration is commonly referred to as an electronic cigarette. The electrical connection with a heating element that elevates the temperature of the e-liquid for such alternate embodiments can be a hardwired connection that is not to be separated and reconnected without damaging the electronic vaporizer. For the sake of brevity and clarity, however, the present technology will be described with reference to the electronic vaping device that includes a separable tank 104 as shown in FIG. 6.

A user interface 124 is provided to the vaporizer body 106, and includes one or a plurality of selectable input devices that offer the user an ability to input commands and optionally user-defined settings that control at least one, and optionally a plurality of parameters of the electronic vaping device. Examples of such parameters include at least one of: (i) an operational mode of the electronic vaping device 100 (e.g., selecting a mode in which the electronic vaping device 100 simulates the yield and/or concentration profile of a tobacco cigarette), (ii) a user-specified power setting for the heating element 114; (iii) a desired vapor temperature setting; and (iv) a quantity setting that defines at least one of: a quantity of a chemical constituent desired to be included in the vapor, and a gas fraction of the chemical constituent in the vapor.

The user interface 124 includes a fire button 126 that, when pressed, causes the controller 102 to initiate a puff by initiating or otherwise controlling the supply of output power from the battery 112 to the heating element 114 as described herein. The heating element 114 is energized by the output power to generate the vapor for the puff, thereby producing the yield and/or concentration profile as described herein.

According to alternate embodiments, the fire button 126 can be replaced by a control routine programmed into a computer processor 128, such as a microcontroller for example, of the controller 102. The control routine can optionally include computer-executable instructions stored in the CRM 130. When executed, the instructions of the control routine can automatically activate the heating element 114 in response to detecting a negative pressure or the flow of air through the tank 104 caused by the user inhaling through the mouthpiece 122. Regardless of how a puff is activated, output power is to be supplied by the battery 112 to the heating element 114 under the control of the controller 102 as described herein.

The user interface 124 can also include a menu button 132, or other suitable data entry device such as a touch-sensitive display, tactile switch, etc. When pressed or otherwise selected, the menu button 132 causes a computer processor 128 of the controller 102 to execute computer-executable instructions stored by the CRM 130 to display one or more menu options on a LED or other suitable display 148. Toggle buttons 150 allow the user to toggle through the menu options.

The embodiment of the controller 102 shown in FIG. 6 also includes a power output component 136. Examples of the power output component 136 can include a DC-DC converter such as a buck and/or boost converter, or other suitable circuit to adjust the power supplied by the battery 112. The power output component 136 can be controlled by a pulse-width modulation signal transmitted by the computer processor 128 to step up and/or step down the voltage supplied by the battery 112 to produce the output power. According to other embodiments, the electric current and/or the voltage supplied by the battery 112 can be controlled in any other manner without departing from the scope of the present disclosure. The output power is controlled to supply the heating element 114 with a suitable output power to cause the electronic vaping device 100 to produce vapor according to the yield and/or concentration profile(s) as described herein.

To measure a quantity indicative of the air flow rate and/or temperature of the vapor flowing through the mouthpiece 122, a sensor 134 is positioned in fluid communication with (e.g., exposed to, or positioned within) an airflow passage 137. For example, the embodiment shown in FIG. 6 shows the sensor 134 (e.g., a pressure sensor, airflow sensor, or other sensor suitable to sense a quantity indicative of the airflow rate) provided to the tank 104. For such an embodiment, the sensor 134 can be disposed in (or exposed to) a portion of the airflow passage 137 between an air inlet 139 and the heating element 114. According to other embodiments, the sensor 134 can be provided to the vaporizer body 106, at a location to sense a quantity of the air flowing to entrain the vapor that is indicative of the airflow rate through the airflow passage 137. The quantity sensed by the sensor 134 can be used by the computer processor 128 to determine the airflow rate flowing over the heating element 114, and through the airflow passage 137.

According to other embodiments, the flow-measuring sensor 134 can include any structure, optionally configured with computer-executable instructions, that is operable to sense or otherwise determine a pressure of the air flowing through the airflow passage. For instance, any pressure-based flow sensor produces an output that can optionally be non-linear with flow, requiring calibration for the specific design and configuration of the airflow passage 137, and/or other structures of the electronic vaping device 100, or portion thereof.

As a specific example, a plurality (e.g., at least two) absolute pressure sensors, or at least one differential pressure sensor, reading on opposite sides of a restrictor plate or at different points in a venturi can constitute an embodiment of the sensor 134 that measures flow rate with sufficient accuracy. For such embodiments, a restriction/venturi could be built into the device itself.

Alternately, to create adequate draw resistance to mimic a cigarette, the electronic vaping device 100 can include one or more inlet openings (whether implicit through leakage or explicit) where the air is drawn from the ambient environment during a puff. Another alternate embodiment for flow sensing is to use this built in inlet (or other geometry inherent to the device) as the restriction or limiter of the airflow through the airflow passage 137. The ambient pressure outside the device can be measured along with the pressure of the air flowing through the airflow passage 137 or another internal passage of the electronic vaping device 100 after the restriction. Such a configuration is less complex than constructing an internal differential measurement, because measuring the ambient pressure isn't as space constrained: atmospheric pressure doesn't change meaningfully from one end (e.g., the top) of the electronic vaping device 100 to the other end (e.g., the bottom) thereof, for example.

According to another example, it can be assumed that ambient pressure does change significantly due to weather, altitude, etc. Further, it can be assumed that ambient pressure does not typically change very fast on an e-cigarette time scale. Weather changes take minutes to hours, for example. In a cost-saving effort, the ambient pressure can optionally be sampled by the sensor 134 when the device is not actively performing a puff or otherwise being puffed on (e.g., the user is not inhaling through the electronic vaping device 100), which is most of the time. Because the electronic vaping device 100, when not in use has both the mouthpiece and the air inlet open to the ambient environment, the pressure in the air/aerosol passage 137 will quickly normalize back to the ambient pressure when the electronic vaping device 100 is not in use. Thus, one pressure sensor can provide reasonably accurate pressure information for both sides of a restrictor plate that limits the permissible inflow of air from the ambient environment. Thus, according to some embodiments, the sensor 134 could simply be configured and positioned on the electronic vaping device 100 to measure the absolute pressure of the air flowing through the airflow passage 137 or other passage of the electronic vaping device 100. A defined value for atmospheric pressure can be assumed, and the defined value can optionally be measured by the sensor 134 (or a separate sensor) when the electronic vaping device is not in use.

For at least one of the above embodiments, the inlet orifice restricting airflow into the electronic vaping device 100 can optionally be fixed, and specified as a known value that is programmed into, or otherwise determined by the controller 102. According to alternate embodiments, however, an adjustable restrictor plate or other structure can be provided to the electronic vaping device 100 to allow a user to set a desired draw resistance to be experienced during a puff. For such embodiments, the sensor 134, or optionally a different sensor in communication with the controller 102 can sense the position or other setting of the restrictor plate, as adjusted by the user. The controller 102 can be programmed to include a plurality of different calibrations of a model, relating the values determined based on the sensor 134 to the air flowing through the airflow passage 137.

Instead of sensing a pressure, alternate embodiments of the sensor 134 can derive airflow values based on operation of a hot wire anemometer. For instance, a true hot wire (or hot plate) anemometer according to such embodiments can include a wire, plate or similar structure that is constructed of a material with properties (resistance, typically) that change with changes in the material's temperature. As another example, a temperature-sensing probe (e.g., thermometer, thermocouple, etc.) can be in thermal communication with such a wire, plate or other structure. In either case, the material is placed into the path of the flow to be measured. The material is heated electrically to a temperature. The amount of energy required to maintain that temperature is equal to the amount of energy being carried away by the flow (plus small conductive or radiative losses that are compensated for during calibration). The amount of energy being carried away by the flow is a function of the flow rate, and the function can be programmed into the controller 102. So by measuring the relationship between the structure temperature and the power required to heat to that temperature, flow rate can be measured (for a given air temperature). Typically this type of system will have a second, unheated temperature sensing structure to measure the air temperature as a reference.

As another example, the temperature of air can optionally be increased by a first coil or other pre-heater prior to introducing the pre-heated air to a second aerosol-generating coil or other heating structure. Using the preheater coil as a hot-wire anemometer requires only a measurement of the inlet or outlet air temperature, measurement of the preheater power and temperature, and a calibration model programmed into the controller 102. The present embodiment is not sensitive to adjustments in the inlet orifice, so for the pre-heated embodiments of the electronic vaping device 100 flow measurement can be added without requiring different controller 102 configurations to account for adjustable restriction of the inlet airflow as described for the embodiments above. Further, sensing the operation or increase in temperature of the heated structure to start the flow of vapor may be avoided for the present embodiments, as the heated structure would have to be maintained at an elevated temperature at all times, negatively impacting battery life. Instead, a pressure switch or a manually-selectable “activate” button, or some other method of sensing use (accelerometer reading device motion, touch sensor on the mouthpiece, etc) to trigger activation of the anemometer can optionally be utilized. In other words, the flow-measuring sensor 134 can include a structure including one or more heated elements and optionally a temperature sensor or module programmed into the controller 102 for relating thermal performance of the heated element to measured airflow.

Other embodiments of the flow sensor 134 can sense airflow values based on the heating element 114 that generates the aerosol, itself. For such embodiments, the heating element 114 can be made from a temperature-sensing material. Electric power from the battery is believed to be consumed according to at least the following mechanisms:
1: heating up the coil, and by conduction the rest of the atomizer;
2: generating vapor from the e-liquid which is carried away by the airflow; and
3: convectively heating the air, vapor and aerosol that passes over the coil.

Such a system can optionally include a heating element 114 configured as a coil wrapped around wicking material 120 in the atomizer. The ends of the coil (touching the electrical contacts) heat up the contacts and the case. The inner half of the heating coil, touching the e-liquid saturated wicking material 120 generates aerosol. The outer half of the coil, touching only air and vapor, convectively heats the air.

The first mechanism is largely independent of the airflow rate, and the second mechanism is independent of the airflow rate up to the point where the air becomes saturated with vapor. The third mechanism can be a function of the airflow rate. For a given power level, the model defining the function establishes that the faster the flow rate, the more convective heat transfer to the air the coil will experience, so the cooler the coil will be.

One factor to take into consideration in the model is that the first mechanism is time-dependent (both at the beginning of the puff and between puffs) so accurate modeling of the thermal mass, thermal resistances and thermal time constants of the atomizer can be programmed into the controller 102. In other words, the relationship between at least the measured resistance or temperature of the aerosol-generating heating element 114 and the power required to achieve that temperature is used by the controller 102 to determine the airflow rate according to the present embodiments. According to some embodiments, the controller 102 can control operation of the heating element 114 based on the airflow rate of the air to at least maintain a temperature of the vapor flowing through the mouthpiece 122 as a flow rate of the air flowing through the airflow passage increases over the relatively-low range of airflow rates.

The description of various embodiments for sensing airflow is not exhaustive, and the claims are not limited to such embodiments. For example, other embodiments can include other flow-measuring structures such as: turbine-based flow sensors, where the air flow would spin a turbine wheel, whose speed would be read via a magnetic, optical or electrical sensor; mechanical airflow sensors; ultrasonic flow sensors; variable area flow sensors, which cause a structure in the airflow passage 137 to move or deform in response to changes in airflow, and the position and/or deformity of the structure is then related to the airflow; vortex flow meters; etc.

As shown in FIG. 4 and discussed above, the concentration (and thus perceived flavor intensity) of traditional electronic vaping devices has a negative slope over the entire range of airflow rates, or over at least a relatively-low range of airflow rates. That is to say, as the user inhales relatively hard the flavor gets less intense than it was when the user inhaled relatively lightly as a result in the substantially flat yield curve, and the influx of increasing quantities of air to dilute the vapor concentration. As shown in FIG. 2 and discussed above, the tobacco cigarette has concentration profile with a positive slope over the respective airflow rates. That is to say, as the user inhales hard (e.g., high airflow rates) the flavor gets more intense than it was when the user inhales light (e.g., low airflow rates). This difference is believed to contribute to the reason why users have to completely re-learn how to smoke when attempting to switch from tobacco cigarettes to e-cigarettes. With a tobacco cigarette, if the experience is too intense, the smoker reduces the intensity of the draw during a puff (e.g., lowers the airflow speed being inhaled) and the flavor lessens. With the traditional electronic vaping device, if the user does the same (overlearned through years of experience with tobacco cigarettes) behavior, the flavor will get more intense. This causes the user to reflexively draw even more slowly, which causes the intensity to increase further. Eventually the intensity increases to a point where the nicotine, other chemical constituent, or the aerosol in whole irritates the user's mouth and throat, which makes them cough.

What is presented herein is a novel electronic vaping device 100 that uses a sensor 134 such as a flow meter, for example, whose output varies with the rate of the flow of air through the airflow passage 137 of the tank 104 to control the output concentration of one or more of the chemical constituents in a predictable fashion. To accurately model a cigarette, a profile of the output concentration (mg/mL) v. airflow rate includes an upward slope for at least some portion of the controllable range (e.g., a slope that is at least less negative than a conventional vaping device—for example, at least −10 mg/ml 3 over the relatively-low range of the airflow rates, or at least a positive slope). According to alternate embodiments, the output yield (mg/sec.) of at least one chemical constituent can be controlled based on the output of the sensor 134 in a predictable fashion to generate a yield v. airflow rate having a positive slope for at least some portion of the controllable range.

According to an illustrative embodiment of the electronic vaping device 100, an analog pressure sensor 134 (e.g., a MEMS pressure sensor) is used in combination with knowledge of the airflow geometry of a portion (e.g., the airflow passage 137, the mouthpiece, 122, etc.) of the electronic vaping device 100 to create a transfer function relating a pressure drop (ambient air to sensor 134) to draw speed (mL/second). This can be accomplished during the development phase using fluid mechanics, computational fluid dynamics, determined empirically with a smoking machine drawing through the electronic vaping device 100 and/or tobacco combustion cigarettes at a variety of airflows, etc. This pressure drop to airflow curve can be stored in the CRM 130 of the controller 102 as a function of the output from the sensor 134, and utilized by the computer processor 128 to calculate the airflow rate through the electronic vaping device 100 at various times, for example 100 times per second.

Also optionally stored in the CRM 130 is a two-dimensional calibration table or other relationship (e.g., algorithm) for relating yield and/or output concentration of the one or more chemical constituents across the operating range of airflows and power to be supplied to the heating element 114. According to some embodiments, the transfer function or pressure drop to airflow curve can be specific to a specific tobacco combustion cigarette. For example, the model can be developed to cause the electronic vaping device 100 to mimic at least one of a quality, quantity and smoking sensation (e.g., intensity) of a specific brand, and optionally type of tobacco combustion cigarette, such as Marlboro Red, Camel Filtered, Camel Unfiltered, etc. If the yield and/or output concentration curves vary across different brands and/or types of tobacco combustion cigarettes, the electronic vaping device 100 can be configured to operate in a manner that results in the generation of yield and/or output concentration curves that closely approximate a brand and/or type of cigarette preferred by the user. Further, the electronic vaping device can be configured to generate temperatures of the vapor inhaled through the mouthpiece 122 that approximately matches the temperatures of cigarette smoke generated by user-preferred brands and/or types of cigarettes, for example.

According to embodiments, the CRM 130 can optionally store a table or other relationship of desirable output concentrations at the various flow rates the electronic vaping device 100 will support. At least a portion, or optionally all of the values in this table, when plotted as output concentration v. airflow rates, produce a curve having a positive slope: that is, as airflow rate increases the desired concentration increases. The slope of the output concentration v. airflow rates can optionally be substantially constant, or optionally exhibit a decreasing slope trend (e.g., the slope lessens with increases in the airflow rate over a portion of the operating range) over the operational range of airflow rates.

According to other embodiments, the CRM 130 can optionally store a table or other relationship of desirable output yield values for various different flow rates the electronic vaping device 100 will support. At least a portion, or optionally all of the values in this table, when plotted as output yield v. airflow rates, produce a curve having a positive slope: that is, as airflow rate increases the desired concentration increases. Further, a complete surface plot for a specific electronic vaping device 100, over a range of at draw speeds (e.g., from 10 to 30 mL/second), and a range of power outputs for the heating element 114 (e.g., from 10 to 15 watts), as shown in FIG. 10, can be stored by the CRM 130.

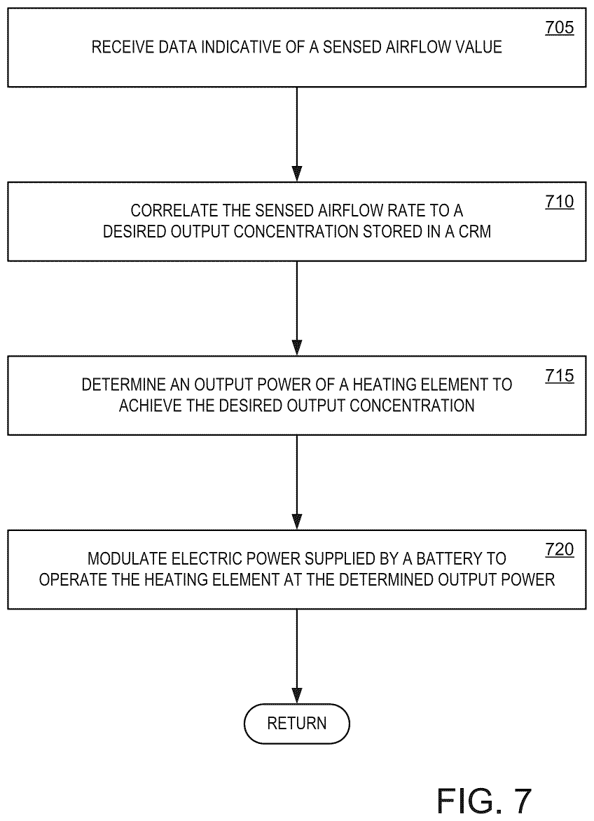

Generally, as shown in FIG. 7, the computing device 128 of the controller 102 receives the output of the sensor 134 indicative of the airflow rate at block 705. Based on this received sensor output, the computing device 128 can access the desired output concentration and/or yield corresponding to the sensed airflow rate stored by the CRM 130 at block 710. An output power to be modulated and supplied to the heating element 114 by the power output component 136 can be obtained at block 715 from the CRM 130 to correspond to the desired concentration. The computing device 128 can control operation of the heating element 114 at block 720 to achieve the desired concentration and/or generate the desired yield, and the process repeated often to accommodate different sensed airflow rates. As a result, a concentration profile (e.g., concentration v. airflow rate) with a positive slope that of a tobacco cigarette can be produce by the electronic vaping device 100, thereby mitigating at least one obstacle to adoption of the electronic vaping device 100 by longtime smokers of tobacco cigarettes.

According to a specific embodiment, from time to time the computer-executable instructions executed by the computing device 128 will read or otherwise receive the pressure or other output from the sensor 134, then use the transfer function to calculate the current airflow rate through the electronic vaping device 100. The computing device 128 interpolates the table of desirable output concentrations to determine the desired output concentration for the currently-sensed airflow rate. The computing device can use bilinear interpolation or other suitable method with the two-dimensional calibration table to determine the output power that will generate that concentration of the chemical constituent, or of the vapor as a whole, for the currently-sensed flow rate. The determined output power setting will be fed as a setpoint update to the power output component 136, causing the output concentration to change to match, or at least approach the desired concentration, even if the airflow rate has changed due to the user inhaling more or less than during a previous iteration.

The power supplied to the heating element 114 from the battery 112 is a primary driver of output yields. In general, the higher the power supplied to the heating element 114, the higher the output yield will be. However, factors beyond the output power also influence the aerosol yield, including the speed of the air drawn though the electronic vaping device 100. High airflow rates can lead to more efficient transport of the vapor out of the device, but can also cause more convective cooling of the heating element or other portion of the atomizer due to convective heat transfer. The data accessed from the CRM 130 takes such factors into consideration.

The electronic vaping device 100 used in the examples set forth herein can include a (qualitatively determined) range of user-selectable useful output settings from 10 to 15 watts, for example, which can be automatically selected by the controller 102 as described herein, instead of being manually set by the user.

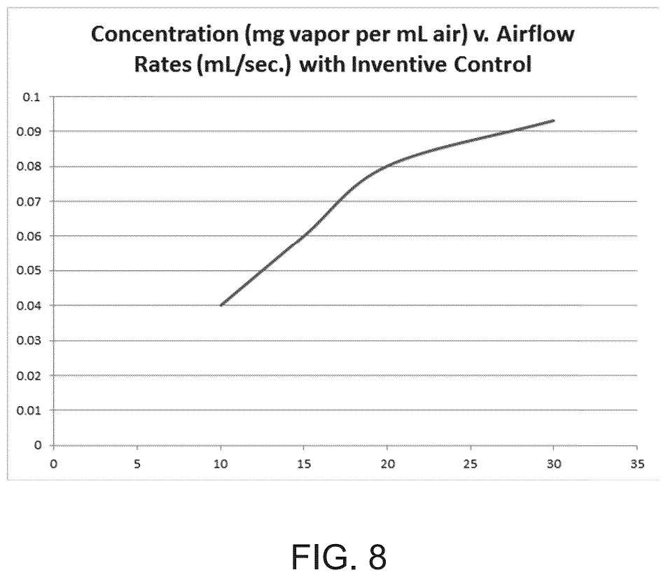

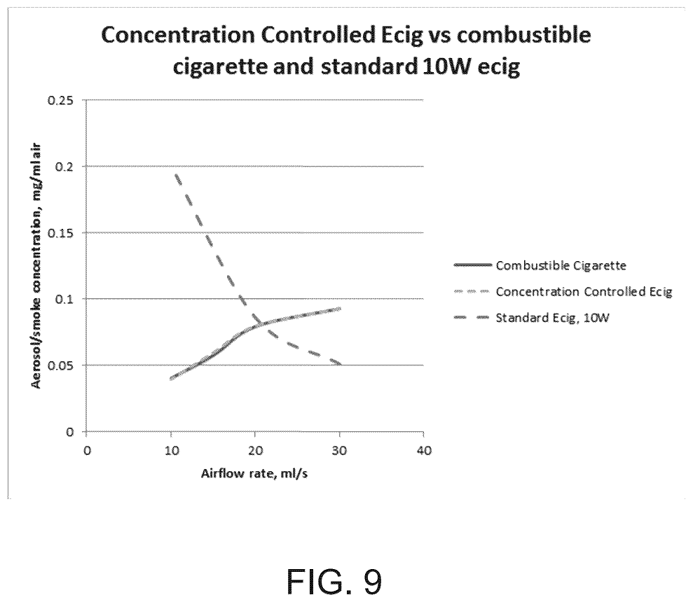

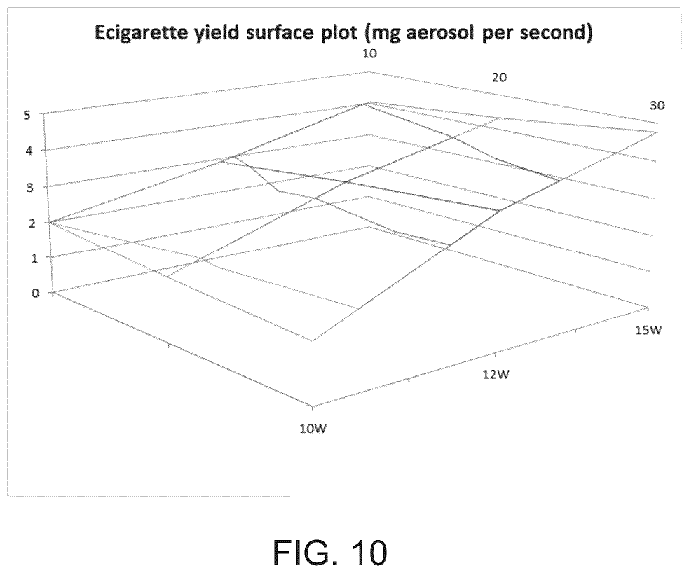

Controlling the electronic vaping device as described herein results in a profile of concentration of at least one chemical constituent, in this example the total vapor, versus airflow rates shown in FIG. 8. A comparison of the concentration profile in FIG. 8 to the concentration of smoke produced by a combustible tobacco cigarette and a conventional electronic vaping device is shown in FIG. 9. The comparison of FIG. 9 shows how well the illustrative embodiment of the control process for the present electronic vaping device 100 coincides with the concentration profile for a combustible tobacco cigarette. A concentration profile of a conventional electronic vaping device, configured similarly to the electronic vaping device 100 but without the inventive controller 102 described herein, set to operate at 10 W of output power is also shown for comparison. As can be seen from FIG. 9, the present electronic vaping device 100 allows the user to take puffs and draw through the electronic vaping device 100 similar to, and optionally the same as the user would a combustible tobacco cigarette at an airflow rate of 20 mL/s. At the same airflow rate, a conventional vaping device achieves a similar instantaneous value of vapor concentration, but this value diverges quickly if the user's airflow rate changes during the puff.

According to some embodiments, because the e-liquid 118 that is vaporized by the electronic vaping device 100 can be manufactured in different nicotine concentrations and flavoring strengths, it is not necessarily desirable to have the magnitude of the output concentration exactly match that of a combustible tobacco cigarette. Similarly, because the electronic vaping device 100 can be manufactured with larger or smaller intended draw volumes and airflow rates, for example high volume “cloud chasing” devices, it may not necessarily be desirable to have the airflow range of the device match the airflow range of a traditional tobacco cigarette as presented here. The disclosed controller 102 can be used to transform any electronic vaping device 100 to have an arbitrary flow rate to output concentration behavior.

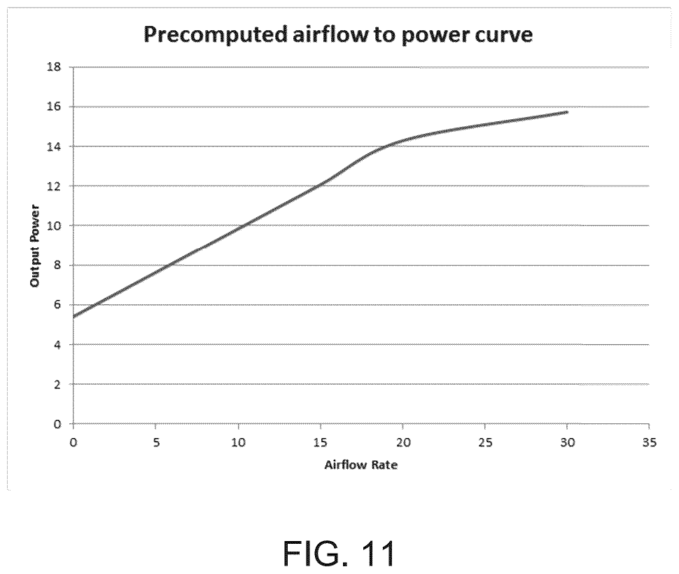

Some users may desire a simple-to-use version of the electronic vaping device 100. For example, an embodiment of a simplified electronic vaping device 100 may offer users one or multiple pre-computed experiences, rather than allowing arbitrary output. For example, a device might have a “light” “regular” and/or “full flavor” mode, corresponding to three airflow-concentration curves. In this case, rather than store the two-dimensional device calibration table in the CRM 130, the transfer function from airflow to output power could be pre-computed or otherwise configured to relate the output power to be supplied to the heating element 114 for ranges of airflow rates for each user setting curve. This saves several on-device computational steps and can be accomplished at lower cost than an electronic vaping device 100 with an available arbitrary power setting. An example of a pre-computed output v. airflow rate profile including a linear region is shown in FIG. 11.

The precomputed curve of FIG. 11 does not necessarily relate an airflow rate to an output power of zero watts. In an electronic vaping device 100 some power is lost to convectively heating the air, some is lost conductively through the wicking material 120, and some is lost in electrical wiring, connectors and the like. Therefore, the most naïve implementation, where output power is directly correlated to airflow or draw pressure without a model, though it might have a positive slope at some point, may not yield results as beneficial to long-terms smokers as the embodiments described above.

An even lower-cost solution would be that for a given sensor, the sensor's raw output (pressure, voltage, resistance, etc.) transfer function could also be premultiplied, or otherwise convolved with model, to give a direct transfer from sensor reading to output power setting.

The desired transfer function need not be taken directly from a combustible cigarette. An e-cigarette designer might create a differently shaped curve to optimize the user experience with a vaping device. As long as there is a meaningful section of ascending concentration in the usable region, it is believed smokers will be able to use the electronic vaping device 100 without coughing or special training.

According to other embodiments, any of a plurality of different flow measuring structures can be utilized instead of, or in addition to a pressure sensor. Pressure-based flowmeters are presented in the example, but any flow measuring structure could be used. Some alternatives are hot wire anemometers, ultrasonic flow meters and turbine wheel flow meters, among others. It is also possible to use the power to resistance or power to temperature correlation of the atomizer heater itself to calculate the airflow rate without an explicit sensor.

Although power control is preferred because it is more consistent, this method can also be used with voltage, current or PWM based output power controllers.

Because the power supplied to the heating element 114 heats the vapor as well as generating the vapor from the e-liquid 118, the output temperature of the vapor can exhibit the same negative slope as the vapor concentration v. airflow rate of a traditional vaping device. For a fixed total output power, more vapor (inlet air plus vapor particles) can lead to lower outlet temperatures. It is possible to apply the same, or similar modeling techniques to make the present electronic vaping device's outlet temperature, rather than concentration, increase with airflow rate, or match or approximate a combustible tobacco cigarette. Temperature can be secondary to concentration in user experience of intensity, so by itself using this technique to control the outlet temperature may not be favored by users. However, for electronic vaping devices that have separate aerosol generating and heating elements, this temperature-based technique can be used to model and control both the vapor (or chemical constituent) concentration and the output vapor temperature simultaneously.

The description herein is focused primarily on the electronic vaping device 100 of FIG. 6. However, the technology described herein is applicable for use with any nicotine-based or other inhaled substance-based electronic cigarette, and any other electronic vaporizer such as wax vaporizers for CBD type products, e-pipes, e-cigars and E-hookas, for example. As an example, the technology described herein is applicable for use with any device that uses electric energy instead of combustion to generate an inhaled vapor.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An electronic vaping device comprising:
a heating element that is to be energized to convert a portion of a medium into a vapor by elevating a temperature of the medium, wherein the medium comprises at least a first chemical constituent to be included in the vapor;
an airflow passage through which air entraining the vapor flows as a result of a user inhaling through a mouthpiece during a puff;
one or more sensors arranged to sense a parameter indicative of an airflow rate of the air entraining the vapor flowing through the airflow passage; and
a controller that controls operation of the heating element based on the airflow rate of the air indicated by the sensed parameter, controlling a concentration of the first chemical constituent based on the airflow rate indicated by the parameter sensed by the one or more sensors.

2. The electronic vaping device of claim 1, wherein the one or more sensors includes at least one of a pressure sensor, hot wire anemometer, and a heating element coil, in fluid communication with the airflow passage.

3. The electronic vaping device of claim 2, wherein the one or more sensors comprise the pressure sensor, and the pressure sensor senses a pressure of the air entraining the vapor.

4. The electronic vaping device of claim 1, wherein the controller controls operation of the heating element to continuously increase a rate at which the first chemical constituent is converted into the vapor over the relatively-low range of the flow rates of the air flowing through the airflow passage.

5. The electronic vaping device of claim 1 further comprising a non-transitory computer-readable medium storing a profile reduceable to concentration that relates a resultant concentration of the first chemical constituent to each of a plurality of different flow rates of the air, wherein:
a relationship between the resultant concentration of the first chemical constituent and the airflow rate of the air flowing through the airflow passage established by the concentration profile exhibits a slope that is at least −0.01 mg/mL³ over the relatively-low range of the airflow rates.

6. The electronic vaping device of claim 5, wherein the slope of the relationship between the resultant concentration of the first chemical constituent and the airflow rate of the air flowing through the airflow passage established by the concentration profile is flat over the relatively-low range of the airflow rates.

7. The electronic vaping device of claim 5, wherein the slope of the relationship between the resultant concentration of the first chemical constituent and the airflow rate of the air flowing through the airflow passage established by the concentration profile is positive over the relatively-low range of the airflow rates.

8. The electronic vaping device of claim 1, wherein the controller further controls operation of the heating element based on the airflow rate of the air indicated by the sensed parameter, at least maintaining a temperature of the vapor flowing through the mouthpiece as a flow rate of the air flowing through the airflow passage increases over the relatively-low range of airflow rates.

9. The electronic vaping device of claim 1, wherein controlling the concentration of the first chemical constituent comprises interfering with dilution of the first chemical constituent as a result of an increase in the airflow rate of the air over a relatively-low range of flow rates through the airflow passage.

10. The electronic vaping device of claim 1, wherein the controller comprises a computer-readable medium storing a model relating operation of the heating element based on the airflow rate to a concentration curve of a specific tobacco combustion cigarette to be simulated by the electronic vaping device.

11. An electronic vaping device comprising:
a heating element that is to be energized to convert a portion of a medium into a vapor by elevating a temperature of the medium, wherein the medium comprises at least a first chemical constituent to be included in the vapor;
an airflow passage through which air entraining the vapor flows as a result of a user inhaling through a mouthpiece during a puff;
one or more sensors arranged to sense a parameter indicative of an airflow rate of the air entraining the vapor flowing through the airflow passage; and
a controller that controls operation of the heating element based on the airflow rate of the air indicated by the sensed parameter, increasing a yield of the first chemical constituent in the vapor entrained in the air as a result of an increase in the airflow rate over a relatively-low range of flow rates of the air flowing through the airflow passage.

12. The electronic vaping device of claim 11, wherein the one or more sensors comprise a pressure sensor in fluid communication with the airflow passage, that senses a pressure of the air entraining the vapor.

13. The electronic vaping device of claim 11, wherein the controller controls operation of the heating element to continuously increase a rate at which the first chemical constituent is converted into the vapor over the relatively-low range of the airflow rates.

14. The electronic vaping device of claim 11 further comprising a non-transitory computer-readable medium storing a profile reduceable to yield that relates a resultant yield of the first chemical constituent to each of a plurality of different values of the airflow rates, wherein:
a relationship between the resultant yield of the first chemical constituent and the airflow rates established by the yield profile exhibits a slope that is not negative over the relatively-low range of airflow rates.

15. The electronic vaping device of claim 14, wherein the slope of the relationship between the resultant yield of the first chemical constituent and the airflow rate of the air flowing through the airflow passage established by the profile reduceable to yield is positive over the relatively-low range of the airflow rates.

16. The electronic vaping device of claim 11, wherein the controller further controls operation of the heating element based on the airflow rate of the air indicated by the sensed parameter, at least maintaining a temperature of the air flowing through the mouthpiece as the airflow rate increases over the relatively-low range of airflow rates.

17. The electronic vaping device of claim 11, wherein the controller further controls operation of the heating element based on the airflow rate of the air indicated by the sensed parameter, interfering with dilution of the first chemical constituent as the airflow rate increases over the relatively-low range of airflow rates.

18. The electronic vaping device of claim 11, wherein the controller comprises a computer-readable medium storing a model relating operation of the heating element based on the airflow rate to a yield curve of a specific tobacco combustion cigarette to be simulated by the electronic vaping device.
